(19) United States
(12) Patent Application Publication
(10) Pub. No.: US 2008/0076180 A1
(43) Pub. Date: Mar. 27, 2008

(54) Method for producing organisms immune to all known viruses and unable to interbreed with other populations by re-encoding their genomes to make use of an alternate genetic code

(76) Inventor: Erik Ethan Eagle Boehm, Milpitas, CA (US)

Correspondence Address: ERIK ETHAN EAGLE BOEHM, 1791 DENNIS AVE., MILPITAS, CA, 95035, US

(21) Appl. No.: 11/534,645

(22) Filed: Sep. 23, 2006

(51) Int. Cl. C12N 15/10; C12N 15/00; C12N 15/01

(52) U.S. Cl. 435/440

(57) ABSTRACT

A method for producing organisms immune to all known viruses and unable to interbreed with other populations by re-encoding their genomes to make use of an alternate genetic code is disclosed. This method involves modifying tRNA anti-codon sequences to recognize a different mRNA codon. The genome of an organism then has all its protein coding regions re-encoded to be compatible with the new tRNA anti-codons. As a result the organism with a re-coded genome will be immune to all current viruses that use variants of the Universal Genetic Code. The organism will also be unable to interbreed with unmodified organisms, due to using a different genetic code.

### BACKGROUND OF THE INVENTION

1) Field of the Invention

This invention relates generally to any organism whose complete genome has been sequenced and annotated, and particularly to important organisms susceptible to significant viral damages, or significant genetic modification.

2) The following describes common knowledge in the field:

The “universal genetic code” is well known to biologists. This code specifies what amino acids will be incorporated into a protein by a cell for a given set of three nucleotides that constitute an mRNA codon. This code is common to nearly all genomes currently found on earth, from Humans, to the Poliovirus, to the diminutive mycoplasm. This code is determined by tRNA adaptor molecules, which carry specific amino acids, and have three specific nucleotides that constitute the anti-codon. The tRNA adaptor molecules serve to match an amino acid to a mRNA codon. They accomplish this by having an anti-codon that binds to an mRNA codon in a sequence specific manner. Each tRNA has a specific amino acid attached to it, and therefore brings the amino acids in the same sequence as specified by the mRNA/DNA.

It is currently possible to commercially order any DNA sequence. Molecules of tRNA are transcribed by cells from DNA, or can be transcribed in vitro from DNA with the use of commercially available enzymes. It is therefore possible to use a DNA sequence to make a tRNA molecule with a corresponding sequence.

There is a small percentage of organisms on this earth that use deviant codes, ones that differ from the standard genetic code by a few codons. This causes problems when their genes are spliced into an organism using the standard code. Experiments with yeast genomes have shown that using different tRNAs could result in different amino acids being put in to a protein. In one such experiment, genes from Candida maltosa were placed in Saccharomyces cerevisiae, it was concluded the CTG codons must be exchanged for TCT to produce normal proteins. This describes taking an alternate genetic code, and re-encoding it for use with the standard genetic code, to produce standard proteins. It should be noted in this case, differing by only one codon from the standard code, “resulted in the formation of still active but unstable enzymes” (A deviation from the universal genetic code in Candida maltosa and consequences for heterologous expression of cytochromes P450 52A4 and 52A5 in Saccharomyces cerevisiae. Yeast. 1995 January; 11(1):33-41.)

Numerous experiments have clearly demonstrated the feasibility of replacing parts of a cells genome, or entire genomes with new DNA sequences.

All viruses are dependent upon machinery of the host cell for replication. As such, to have their nucleic acids translated into protein, they must make use of the tRNAs of the host cell. As a result, it is a requirement that a virus use the same method of encoding amino acid sequences into nucleic acid sequences as the host cell.

A Genetically Modified Organism (GMO) present many possible benefits. However, there is a great concern about the environmental impact of such GMOs, as there is a potential for the GMO to interbreed with wild populations and spread harmful genes.

### BRIEF SUMMARY OF THE INVENTION

A method for producing organisms immune to all known viruses and unable to interbreed with other populations by re-encoding their genomes to make use of an alternate genetic code is disclosed consisting of generating a genetic code for specifying amino acids that is incompatible with the standard genetic code or variants thereof, modification of the DNA of an organism such that its tRNAs can correctly mediate the aforementioned code, and re-encoding the protein encoding regions the aforementioned organism.

The advantages and further details of the present invention will become apparent in the following detailed description.

### BRIEF DESCRIPTION OF THE SEVERAL VIEWS OF THE DRAWING

FIG. 1—The universal Genetic code, as found in organisms worldwide. It codes for specific amino acids based on the mRNA code

FIG. 2—A possible genetic code to effect viral immunity. This code would map enough different amino acids to different mRNA codons, making viral genomes incompatible with it.

### DETAILED DESCRIPTION OF THE INVENTION

This invention is a way to effect viral immunity by encoding a genome to operate under a different encoding for amino acid sequences.

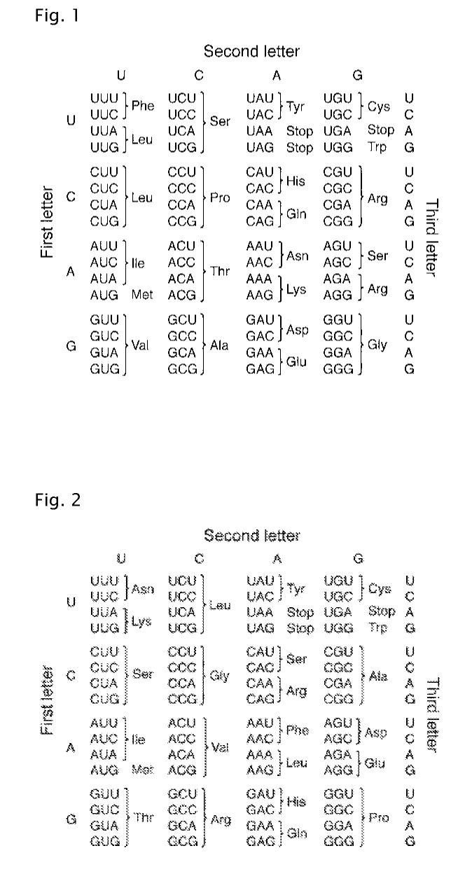

The current Universal Genetic code is seen in FIG. 1. This invention first involves generating a new genetic code that is incompatible with the Universal Genetic code.

As previously noted in the Candida maltosa example, merely changing one codon was not enough to produce completely non-functional proteins. As such, there is a chance that many viruses would still be able to replicate inside a cell with only a few non-standard tRNAs. Additionally, changing a codon to code for similar amino acid should be avoided, i.e. the Leucine codons should not be changed to code for Isoleucine. Substituting one hydrophobic amino acid for another should be avoided to ensure incompatibility, as should substituting one nitrogen-containing amino acid for another.

The new code makes enough codons specify a different amino acid than found under the Universal Genetic Code, such that a virus using the universal genetic code or variant there of is guaranteed to produce defective proteins.

An example of an alternate way of encoding protein sequence is seen in FIG. 2. This code changes nearly all the codons, and will result in incompatibility with current viral genomes.

The new code is to be compatible with every aspect of the organism it is to be applied to, with the exception of some tRNAs. As initiation and start codons involve direct interactions with molecules other than tRNAs, they remain unchanged. Additionally the third base pair of tRNAs are often modified, so a new codon for an amino acid uses the same base pair at the third nucleotide.

A system that uses the new encoding is needed. To accomplish this modified tRNAs are used. This involves identifying the tRNA sequences that encode for the amino acids that are to be encoded differently. Next the 3 consecutive nucleotides that comprise the anti-codon are identified. The tRNA anti-codon sequences are then changed to a sequence that corresponding to the new genetic code such that it can correctly base pair with its corresponding codon and insert the correct amino acid. The corresponding DNA molecule is then is then synthesized and used to produce the corresponding tRNAs by standard processes.

To elaborate with an example, under the universal genetic code seen in FIG. 1, a tRNA carrying Phenylalanine with the anti-codon GAA will recognize the Phenylalanine mRNA codon UUC. This GAA anti-codon must be changed to GTT under the new coding system shown in FIG. 2, such that the tRNA will instead insert Phenylalanine amino acids into the sequence when the new Phenylalanine mRNA codons AAU or AAC are encountered.

Alternately, a tRNA may retain the same anti-codon, but be modified in such a way that it becomes charged with a different amino acid. It does not matter which process is used, as long as the tRNAs pair different amino acids to different codons, as specified per the previously described new code.

The next step makes the nucleic acid sequence/genome of the target organism compatible with the new protein encoding method. This involves changing the original codons within the protein encoding regions to the new one codons specified by the new code, as well as the genes for the tRNAs that will read the new codons, such that the DNA encodes the same amino acid sequence.

Due to the large size of genomes, the only current practical way to re-encode genomes would is via a computer program. This program maps all the original codons within an open reading frame to a new codon as specified by the new code for every protein encoding region within the genome.

Elaborating with an example: Using the code in FIG. 2 this mapping would leave every AUG codon unchanged, but would change every UUU codon to a TTU codon; it would change every UUC codon to a TTCcodon.

The corresponding DNA sequences are then be synthesized in a commercial lab.

For large genomes consisting mainly of non-coding regions, it may not be practical to re-synthesize the entire genome with just the protein coding regions changed. In this case splicing out just the old protein and tRNA coding regions with the new re-encoded ones by standard methods creates the new genome. This approach would is much cheaper for organisms with large genomes that are mostly non-coding, but both are be viable.

Given the large numbers of homologous protein encoding genes (especially metabolic genes), once an initial organism is re-encoded, much of the DNA for the re-encoded genes can be used when re-encoding the genome of another species, thus considerably lowering the cost of successive attempts at re-encoding genomes to the new code.

What is claimed is:

1. Generation of a genetic code for specifying amino acids that uses sufficiently different codons for amino acids, such that existing genomes will be incompatible with it and produce non functional proteins, while not significantly interfering with other cell processes not related to encoding amino acids.

2. Modification of tRNA anti-codons sequences, such that codons are associated with amino acids in a significantly different way than found in unmodified organisms, such that usage of the tRNA sequences would be guaranteed to produce non-functional proteins for unmodified genomes.

3. The Re-encoding of the DNA protein encoding sequences of a genome for use with a new genetic code that is incompatible with current viruses, such that a set of modified tRNA molecules will be able to correctly mediate the genetic code translation and produce functional proteins.
